(19) United States
(12) Patent Application Publication
(10) Pub. No.: US 2023/0157753 A1
(43) Pub. Date: May 25, 2023

(54) SYSTEMS AND METHODS FOR TATTOO REMOVAL

(71) Applicant: Marci White, Scottsdale, AZ (US)

(72) Inventor: Marci White, Scottsdale, AZ (US)

(21) Appl. No.: 17/817,167

(22) Filed: Aug. 3, 2022

Related U.S. Application Data
(60) Provisional application No. 63/281,545, filed on Nov. 19, 2021.

(51) Int. Cl. A61B 18/20; A61M 37/00

(52) U.S. Cl. CPC A61B 18/203; A61M 37/0015; A61B 2018/2065; A61B 2017/00769

(57) ABSTRACT

Systems and methods for tattoo removal according to various aspects of the present technology may comprise performing: a first treatment using a first multi-wavelength laser; a second treatment using a second multi-wavelength laser; a third treatment using a microneedling device; and a fourth treatment using a chemical solution.

### CROSS REFERENCE TO RELATED APPLICATIONS

This application claims the benefit of U.S. Provisional Patent Application Ser. No. 63/281,545, filed on November 19, 2021, and incorporates the disclosure of the application in its entirety by reference.

### BACKGROUND OF THE TECHNOLOGY

### State of the Art

Despite the widespread acceptance and popularity of tattoos, there is also a growing demand among various demographic groups for the complete or partial removal of tattoos. Various systems and methods exist to remove unwanted tattoos. One such system and method is a laser-based system and method.

Conventional laser-based methods for tattoo removal generally involve passing a laser light over a tattoo, heating and fracturing the tattoo ink particles. However, as the laser light passes over the tattoo, it often penetrates the skin in an invasive manner, which can lead a recipient of the laser, i.e., a patient, to develop a variety of skin complications, such as reddening, unwanted pigmentation, including hyper pigmentation, scarring, and even bubbling. In addition, conventional laser-based methods for tattoo removal often require numerous treatment sessions to penetrate through all of the varying depths of ink in the tattoo, thereby further exacerbating the complications.

Accordingly, what is needed is a system and method for removing unwanted tattoos that efficiently treats a wide range of tattoo colors across a broad range of skin types, does not require pre-laser anesthesia, leaves the skin intact, and reduces or eliminates complications with little to no downtime.

### SUMMARY OF THE TECHNOLOGY

Systems and methods for tattoo removal according to various aspects of the present technology may comprise performing: a first treatment using a first multi-wavelength laser; a second treatment using a second multi-wavelength laser; a third treatment using a microneedling device; and a fourth treatment using a chemical solution.

### BRIEF DESCRIPTION OF THE DRAWINGS

A more complete understanding of the present technology may be derived by referring to the detailed description and claims when considered in connection with the following illustrative figures. In the following figures, like reference numbers refer to similar elements and steps throughout the figures.

FIG. 1 is a block diagram of a system for tattoo removal in accordance with an embodiment of the present technology; and

FIG. 2 representatively illustrates a flow diagram for performing a method for tattoo removal in accordance with an embodiment of the present technology.

### DETAILED DESCRIPTION OF EMBODIMENTS

The present technology may be described in terms of functional block components. Such functional blocks may be realized by any number of components configured to perform the specified functions and achieve the various results. For example, the present technology may employ various lasers, microneedling devices, chemical solutions, and the like, which may carry out a variety of functions. In addition, the present technology may be practiced in conjunction with any one of various systems for tattoo removal, and the system and method described herein is merely one exemplary application for the technology.

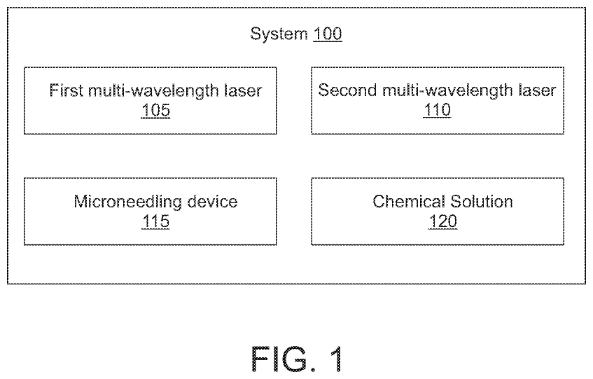

Referring to FIG. 1, a system and method for tattoo removal according to various aspects of the present technology may be representatively illustrated by a system 100 for use on a patient by any suitable practitioner, such as a nurse, nurse practitioner, physician, physician assistant, dermatologist, plastic surgeon, cosmetic surgeon, aesthetician, and the like. According to various embodiments, the system 100 may comprise a first multi-wavelength laser 105, a second multi-wavelength laser 110, a microneedling device 115, and a chemical solution 120.

The first multi-wavelength laser 105 may be suitably configured to target ink particles located in the exodermis layer of the patient's skin. In particular, the first multi-wavelength laser 105 may be a fractional laser. In the context of this Application, the term “fractional laser” means a laser that may be utilized by a practitioner to treat or affect a fraction or portion of a patient's skin, i.e., target area, while leaving the healthy skin cells around the target area unharmed.

In one embodiment, the first multi-wavelength laser 105 may comprise an ultrashort-pulse (USP) laser capable of emitting one or more streams of ultra-short high-energy pulses. Specifically, each pulse may be absorbed by the tattoo ink particles. As the tattoo ink particles absorb the pulses, and thus the energy, they may shatter into tiny fragments. In this regard, over time, the patient's immune system may remove the tattoo ink particles from the target area, lightening and thereafter quickly eliminating the appearance of the tattoo. As an example, the first multi-wavelength laser 105 may comprise a picosecond laser, such as a PicoWay Zoom™ laser. Because the first multi-wavelength laser 105 may be fast and powerful, it may not cause the outer layers of the patient's skin to break, so the patient may enjoy little to no downtime after treatment. It will, however, be appreciated that modifications may be made to the first multi-wavelength laser 105 without departing from the scope of the present invention. For example, instead of a picosecond laser, a femtosecond laser or any other suitable laser may be used.

In various embodiments, the first multi-wavelength laser 105 may be configured to emit a first stream of pulses, a second stream of pulses, a third stream of pulses, and a fourth stream of pulses. The first stream of pulses may comprise a first wavelength; the second stream of pulses may comprise a second wavelength; the third stream of pulses may comprise a third wavelength; and the fourth stream of pulses may comprise a fourth wavelength. The first, second, third, and fourth wavelengths may be selected according to the patient's skin type, tattoo color, or the like. For example, the first wavelength may be 785 nm and may be utilized for targeting black, blue, purple, and green tattoo ink as well as a variety of other tattoo inks. The second wavelength may be 730 nm and may be utilized for targeting green, blue, and purple tattoo ink as well as a variety of other tattoo inks. The third wavelength may be 1064 nm and may be utilized for targeting black tattoo ink as well as a variety of other dark tattoo inks including, but not limited to, blue and purple. The fourth wavelength may be 532 nm and may be utilized for targeting yellow, red, and warm toned tattoo inks including orange and brown tattoo ink.

The second multi-wavelength laser 110 may be suitably configured to target scars, wrinkles, pigmentation, including hyper pigmentation, and other imperfections, located in a second layer of the patient's skin, i.e., dermis, while leaving the outermost layer of the patient's skin, i.e., epidermis, intact. In one embodiment, the second multi-wavelength laser 110 may comprise an ultrashort-pulse (USP) laser capable of emitting a plurality of ultra-short high-energy pulse streams. In particular, the second multi-wavelength laser 110 may be a fractional laser and may be capable of emitting a plurality of ultra-short high-energy pulse streams that are spaced evenly apart when applied to a fraction or portion of the patient's skin. As an example, the second multi-wavelength laser 110 may comprise a picosecond laser, such as a PicoWay Resolve™ laser. In this regard, the second multi-wavelength laser 110 may minimize or prevent damage to the patient's skin. It will, however, be appreciated that modifications may be made to the second multi-wavelength laser 110 without departing from the scope of the present invention. For example, instead of a picosecond laser, a femtosecond laser or any other suitable laser may be used.

In one embodiment, the second multi-wavelength laser 110 may be configured to emit a first plurality of pulse streams and a second plurality of pulse streams. Each of the first plurality of pulse streams may comprise a fifth wavelength, and each of the second plurality of pulse streams may comprise a sixth wavelength. The fifth and sixth wavelengths may be selected according to the patient's skin type, type of imperfection, or the like. For example, in one embodiment, the fifth wavelength may be 532 nm and may be utilized for treating a variety of skin imperfections, such as redness, tightness, texture, pigmentation, including hyper pigmentation, and the like. The sixth wavelength may be 1064 nm and may be utilized for treating deeper imperfections such as scars, etc. that trap tattoo ink.

The microneedling device 115 may comprise any minimally invasive, non-ablative, and (in some cases) non-surgical device suitably configured to pierce the skin of the patient in order to trigger new collagen synthesis without causing scar tissue formation. For example, in one embodiment, the microneedling device 115 may comprise a plurality of needles (not shown) arranged in a pattern on the tip of a pen-shaped instrument (not shown) and coupled to a motor (not shown) located inside the pen-shaped instrument (not shown). In particular, the plurality of needles may be repeatedly, and in some cases rapidly, inserted and removed or withdrawn from the skin of the patient, causing tiny puncture wounds as they press against and/or are inserted into the skin. This process may trigger the release of growth factors and cytokines, producing more collagen and elastin to stimulate the skin's natural ability to repair itself, resulting in smooth, plump, and younger looking skin. This process also breaks down scar tissue, which holds tattoo ink in place and prevents the patient's immune system from removing the ink particles after a fractional laser has previously targeted the tattoo ink.

It will be appreciated that modifications may be made to the microneedling device 115 without departing from the scope of the present invention including RF (radio frequency). For example, in one embodiment, the plurality of needles (not shown) may be coupled to a cylindrical-shaped component (not shown) that may be rolled across the skin. In an alternative embodiment, the plurality of needles (not shown) may project from a flat surface (not shown) attached to a handle (not shown) such that the plurality of needles (not shown) may be “stamped” on the skin. It will also be appreciated that instead of needles (not shown), tips (not shown) or pins (not shown) may be used.

According to various embodiments, the chemical solution 120 may comprise any suitable solution capable of being used in a “chemical peel” treatment. For example, the chemical solution 120 may comprise hyaluronic acid, glycolic acid, trichloroacetic acid, salicylic acid, lactic acid, carbolic acid (phenol), or any combination thereof In particular, the chemical solution 120 may be applied to the patient's skin at different depths, from light to deep. When the chemical solution is applied to the skin, it may raise the acidity of the skin to a pH level of around 3.8. Because skin generally has a pH of around 5.5, changing the pH level may loosen the cells that connect the dead skin and healthy skin, thereby allowing the dead skin to peel off. After the dead skin peels off, new healthy skin may grow back smoother and less wrinkled and pigmented than the old skin.

In operation, an efficient method for tattoo removal and treating skin may comprise performing: a first treatment using the first multi-wavelength laser 105; a second treatment using the second multi-wavelength laser 110; a third treatment using the microneedling device 115; and a fourth treatment using the chemical solution 120.

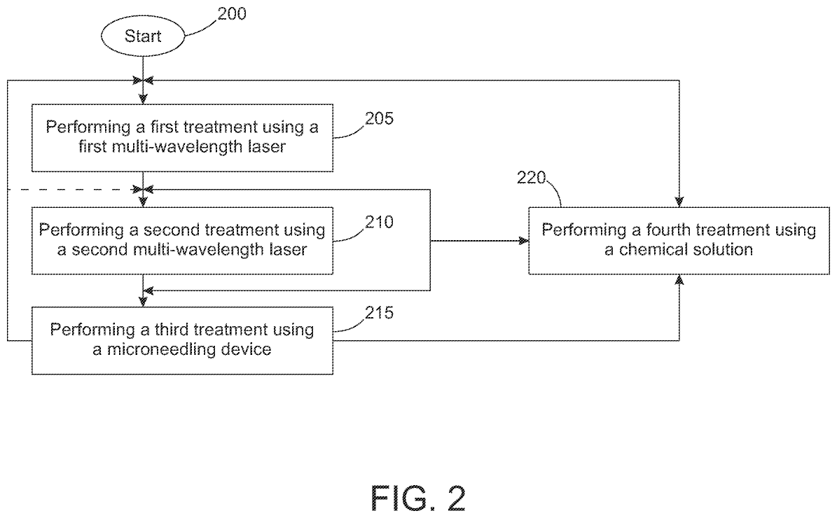

Referring now to FIGS. 1-2, at the start (200), a practitioner may begin by cleaning and/or disinfecting the outermost layer of the patient's skin and subsequently marking a fraction or portion, i.e., target area, on the outermost layer. The target area may include the entire tattoo or a portion of the tattoo.

After the start (200), the practitioner may begin performing the first treatment (205). Performing the first treatment (205) may comprise using the first multi-wavelength laser 105 to apply the first stream of pulses and the second stream of pulses to the target area. Specifically, the practitioner may guide the first stream of pulses and the second stream of pulses over the target area. As discussed in paragraph [0012] of this Application, the first stream of pulses may comprise a first wavelength of 785 nm, which may be utilized for targeting black, blue, purple, and green tattoo ink as well as a variety of other tattoo inks. As also discussed in paragraph [0012] of this Application, the second stream of pulses may comprise a second wavelength of 730 nm, which may be utilized for targeting green, blue, and purple tattoo ink as well as a variety of other tattoo inks. The first stream of pulses may be applied to the target area prior to the second stream of pulses. Alternatively, the second stream of pulses may be applied to the target area prior to the first stream of pulses.

After the first stream of pulses and the second stream of pulses are applied to the target area, performing the first treatment (205) may further comprise using the first multi-wavelength laser 105 to apply the third stream of pulses and the fourth stream of pulses to the target area. Specifically, the practitioner may guide the third stream of pulses and the fourth stream of pulses over the target area. As discussed in paragraph [0012] of this Application, the third stream of pulses may comprise a third wavelength of 1064 nm and may be utilized for targeting black tattoo ink as well as a variety of other dark tattoo inks including, but not limited to, blue and purple. As also discussed in paragraph [0012] of this Application, the fourth stream of pulses may comprise a fourth wavelength of 532 nm and may be utilized by the practitioner to target yellow, red, and warm toned tattoo inks, such as orange and brown. The third stream of pulses may be applied to the target area prior to the fourth stream of pulses. Alternatively, the fourth stream of pulses may be applied to the target area prior to the third stream of pulses. The first treatment may take around 20 to 60 minutes to complete.

It will be appreciated that modifications may be made to the number of wavelengths used and order in which the streams of pulses are applied to the target area without departing from the scope of the present invention. Specifically, the first stream of pulses, second stream of pulses, third stream of pulses, and fourth stream of pulses may be applied to the target area in any order.

After the first treatment is performed, performing the method for tattoo removal may also comprise performing the second treatment (210). Performing the second treatment (210) may comprise using the second multi-wavelength laser 110 to apply the first plurality of pulse streams and the second plurality of pulse streams to the target area. As discussed in paragraph [0014] of this Application, each of the first plurality of pulse streams may comprise a fifth wavelength of 532 nm, which may be utilized by the practitioner to treat a variety of skin imperfections, such as redness, tightness, texture, pigmentation, including hyper pigmentation, and the like. As also discussed in paragraph [0014] of this Application, each of the second plurality of pulse streams may comprise a sixth wavelength of 1064 nm, which may be utilized by the practitioner to treat a variety of deeper skin imperfections including scars. In particular, the second plurality of pulse streams may be utilized by the practitioner to remodel scar tissue to allow trapped ink to escape and soften the appearance of scars.

The first plurality of pulse streams may be applied to the target area prior to the second plurality of pulse streams. Alternatively, the second plurality of pulse streams may be applied to the target area prior to the first plurality of pulse streams. The second treatment may take the practitioner around 15 to 20 minutes to complete and may be performed immediately after the first treatment, a few minutes after the first treatment, a few hours after the first treatment, a few days after the first treatment, or even several weeks after the first treatment. In addition, the first treatment and the second treatment may each be repeatedly performed every six (6) to eight (8) weeks until none of the tattoo ink and/or skin imperfections remain.

Before or after the second treatment is performed, performing the method for tattoo removal may further comprise performing the third treatment (215). Performing the third treatment (215) may comprise utilizing the microneedling device 115 to pierce the target area in a minimally invasive manner to stimulate the skin's natural ability to repair itself by triggering collagen and elastin production in the skin, preventing post laser treatment complications and aiding in the removal of tattoo ink. The third treatment may take the practitioner around 20 to 30 minutes to complete and may be performed between a first session of the first treatment and the second treatment and repeated sessions of the first treatment and the second treatment. In addition, the third treatment may be repeatedly performed every four (4) to six (6) weeks until none of the tattoo ink and/or skin imperfections remain.

In addition, performing the third treatment (215) may comprise collecting a sample of the patient's blood after utilizing the microneedling device 115. Specifically, the practitioner performing the third treatment may isolate protein-rich plasma and platelets from the patient's blood sample to create platelet-rich plasma, i.e., PRP, which may comprise a variety of growth factors capable of promoting skin rejuvenation. The patient's blood may be processed and separated into PRP in any suitable manner, such as by utilizing a centrifuge or the like. Performing the third treatment (215) may further comprise applying the PRP to the target area prior to microneedeling to push the PRP deeper into the skin. After the PRP is applied to the target area, it may expedite the skin's natural ability to repair itself by releasing a variety of growth factors. As an example, the practitioner may perform a “PRP Liquid Gold Treatment.” Alternatively, the practitioner may utilize hyaluronic acid, glycolic acid, trichloroacetic acid, salicylic acid, lactic acid, carbolic acid (phenol), or any combination thereof to the target area prior to microneedeling in order to push the product deeper into the skin.

Performing the method for tattoo removal may further comprise performing the fourth treatment (220). Performing the fourth treatment (220) may comprise applying a chemical solution, such as hyaluronic acid, glycolic acid, trichloroacetic acid, salicylic acid, lactic acid, carbolic acid (phenol), or any combination thereof to the target area. As discussed in paragraph [0017] of this Application, after the chemical solution is applied to the target area, it may raise the acidity of the patient's skin to a pH level of around 3.8. Because skin generally has a pH of around 5.5, changing the pH level may loosen the cells that connect the dead skin and healthy skin, thereby allowing the dead skin to peel off. After the dead skin peels off, new healthy skin may grow back smoother and less wrinkled and pigmented than the old skin. The fourth treatment may be performed at any time during the treatment process and may be repeatedly performed every four (4) to six (6) weeks until the patient's skin is smooth, plump, and younger looking.

It will be appreciated that modifications may be made to the order in which the various treatments are performed without departing from the scope of the present invention. For example, the first treatment may be performed prior to the second treatment or vice versa. In addition, the third treatment may be performed between the first treatment and the second treatment. Alternatively, the third treatment may be performed at any other suitable time, such as prior to the first treatment and the second treatment or after the first treatment and the second treatment. As discussed in paragraph [0025] of this Application, the fourth treatment may be performed at any time. Furthermore, the first treatment, second treatment, third treatment, and fourth treatment may each be repeatedly performed any number of times until the desired results are obtained.

The particular implementations shown and described are illustrative of the technology and its best mode and are not intended to otherwise limit the scope of the present technology in any way. Indeed, for the sake of brevity, conventional manufacturing, connection, preparation, and other functional aspects of the system and method may not be described in detail. Furthermore, the connections and points of contact shown in the various figures are intended to represent exemplary physical relationships between the various elements. Many alternative or additional functional relationships or physical connections may be present in a practical system.

Embodiments of the present technology may be used in conjunction with any number of methods and devices for tattoo removal. In the foregoing description, the technology has been described with reference to specific exemplary embodiments. Various modifications and changes may be made, however, without departing from the scope of the present technology as set forth. The description and figures are to be regarded in an illustrative manner, rather than a restrictive one and all such modifications are intended to be included within the scope of the present technology. Accordingly, the scope of the technology should be determined by the generic embodiments described and their legal equivalents rather than by merely the specific examples described above. For example, the steps recited in any method or process embodiment may be executed in any appropriate order and are not limited to the explicit order presented in the specific examples. Additionally, the components and/or elements recited in any system embodiment may be combined in a variety of permutations to produce substantially the same result as the present technology and are accordingly not limited to the specific configuration recited in the specific examples.

Benefits, other advantages, and solutions to problems have been described above with regard to particular embodiments. Any benefit, advantage, solution to problems or any element that may cause any particular benefit, advantage, or solution to occur or to become more pronounced, however, is not to be construed as a critical, required, or essential feature or component.

The terms “comprises,” “comprising,” or any variation thereof, are intended to reference a non-exclusive inclusion, such that a system, process, method, article, composition, or apparatus that comprises a list of elements does not include only those elements recited but may also include other elements not expressly listed or inherent to such system, process, method, article, composition, or apparatus. Other combinations and/or modifications of the above-described structures, arrangements, applications, proportions, elements, materials, or components used in the practice of the present technology, in addition to those not specifically recited, may be varied, or otherwise particularly adapted to specific environments, manufacturing specifications, design parameters or other operating requirements without departing from the general principles of the same.

The present technology has been described above with reference to an exemplary embodiment. However, changes and modifications may be made to the exemplary embodiment without departing from the scope of the present technology. These and other changes or modifications are intended to be included within the scope of the present technology.

1. A method for removing a tattoo from a target area of skin using a first multi-wavelength laser, a second multi-wavelength laser, a microneedling device, and a chemical solution, comprising:
performing a first treatment using the first multi-wavelength laser;
performing a second treatment using the second multi-wavelength laser;
performing a third treatment using the microneedling device; and
performing a fourth treatment using the chemical solution.

2. The method of claim 1, wherein performing the first treatment process comprises applying a first stream of pulses, a second stream of pulses, a third stream of pulses, and a fourth stream of pulses to the target area, and wherein:
the first stream of pulses comprises a first wavelength;
the second stream of pulses comprises a second wavelength;
the third stream of pulses comprises a third wavelength; and
the fourth stream of pulses comprises a fourth wavelength.

3. The method of claim 2, wherein:
the first wavelength is 785 nm;
the second wavelength is 730 nm;
the third wavelength is 1064 nm; and
the fourth wavelength is 532 nm.

4. The method of claim 1, wherein performing the third treatment process comprises:
piercing the target area to stimulate collagen production in the skin;
puncturing the target area to collect a blood sample;
utilizing a centrifuge to form a platelet-rich plasma from the blood sample; and
applying the platelet-rich plasma to the target area.

5. The method of claim 4, wherein performing the third treatment process further comprises repeatedly inserting and removing a plurality of needles of the microneedling device from the skin after applying the platelet-rich plasma to the target area.

6. The method of claim 1, wherein performing the fourth treatment process comprises applying the chemical solution to the target area.

7. The method of claim 6, wherein the chemical solution comprises hyaluronic acid, glycolic acid, trichloroacetic acid, salicylic acid, lactic acid, carbolic acid, or a combination thereof.

8. The method of claim 1, wherein:
the first treatment process is performed prior to the second treatment process;
the second treatment process is performed prior to the third treatment process; and
the fourth treatment process is performed after the third treatment process.

9. The method of claim 8, wherein the fourth treatment process is applied prior to the first treatment process.

10. The method of claim 8, wherein the fourth treatment process is performed between the first treatment process and the second treatment process.

11. The method of claim 8, wherein the first treatment process and the second treatment process are each repeatedly performed every six to eight weeks.

12. The method of claim 8, wherein the third treatment process and the fourth treatment process are each repeatedly performed every four to six weeks.
